(19) United States
(12) Patent Application Publication
(10) Pub. No.: US 2018/0036362 A1
(43) Pub. Date: Feb. 8, 2018

(54) TECAP Liquid

(71) Applicant: Tasha Allen, Wesley Chapel, FL (US)

(72) Inventor: Tasha Allen, Wesley Chapel, FL (US)

(21) Appl. No.: 15/666,541

(22) Filed: Aug. 1, 2017

Related U.S. Application Data
(60) Provisional application No. 62/370,859, filed on Aug. 4, 2016.

(51) Int. Cl. A61K 36/48; A61K 35/32; A61K 36/21; A61K 36/28; A61K 36/45; A61K 36/752; A61K 36/185; A61K 36/31; A61K 36/23; A61K 36/42; A61K 31/194; A61K 31/19; A61K 31/047; A23L 33/00; A23L 2/52; A61K 35/12

(52) U.S. Cl. CPC A61K 36/48; A61K 35/12; A61K 35/32; A61K 36/21; A61K 36/28; A61K 36/45; A61K 36/752; A61K 36/185; A61K 36/31; A61K 36/23; A61K 36/42; A61K 31/194; A61K 31/19; A61K 31/047; A23L 33/40; A23L 2/52; A23V 2002/00

(57) ABSTRACT

The present disclosure relates to a natural product drink formula and method, which safely aids in the balancing of vaginal pH, reducing vaginal odor, and/or reducing body odor of the user. The present disclosure may comprise natural ingredients including vitamins and minerals. The present disclosure may be in the form of a powder, a gel, a solid, or a liquid which is later added to water or can be make directly as a liquid drink.

### RELATED APPLICATION

The present application claims priority to U.S. provisional patent application No. 62/370,859 filed Aug. 4, 2016 the entire contents of which are incorporated herein by reference.

### FIELD OF THE INVENTION

The present invention relates generally to a drink formula. More specifically, the present invention is a liquid combination of natural products, juices and vitamins that alleviates or helps prevent vaginal odor, body odor and/or balances the internal pH of the user.

### SUMMARY

The present disclosure relates to a natural product drink and method of use thereof.

### BACKGROUND OF THE INVENTION

Foul odors can be caused by certain medications, hormones, excessive perspiration, sexual intercourse, consumption of a malodorous meal such as seafood, and semen. It is an object of the present invention to alleviate or help prevent these foul odors as well as balance the internal pH of the user through the ingestion of a liquid combination of juices and vitamins.

### BRIEF DESCRIPTION OF THE DRAWINGS

A more complete understanding of the present invention is derived by referring to the detailed description and claims when considered in connection with the Figures, wherein like reference numbers refer to similar items throughout the Figures.

FIG. 1 illustrates a natural product formulation.

Elements and steps in the figures are illustrated for simplicity and clarity and have not necessarily been rendered according to any particular sequence. For example, steps that are performed concurrently or in different order are illustrated in the figures to help improve understanding of embodiments of the present invention.

### DETAILED DESCRIPTION

The following description is provided to enable any person skilled in the art to make and use the invention and sets forth the best modes contemplated by the inventor of carrying out his invention. Various modifications, however, will remain readily apparent to those skilled in the art, since the general principles of the present invention have been defined herein specifically to provide a natural product drink for balancing vaginal pH, reducing vaginal odor, and/or reducing body odor.

Embodiments consistent with the present invention address the need for an efficient vaginal pH formula and method that provides a safe and reliable means of reducing vaginal odor and/or body odor for women and the general public. The present invention is described with regard to a drink formula.

The present disclosure relates to a liquid combination of juices and vitamins that alleviates or helps prevent vaginal odor, body odor and/or balances the internal vaginal pH of the user. Normal vaginal pH ranges from 3.5 to 4.5 and when the vaginal pH becomes imbalanced vaginal odor may result. The user would take a small amount daily, two times a day, right after sexual intercourse, or before consumption of a malodorous meal.

In one embodiment, one or more natural product constituents are combined with a solvent to form the natural product drink. The natural product drink aids in (1) balancing the vaginal pH of the user, (2) reducing vaginal odor, and/or (3) reducing body odor. For example, the natural product drink aids in balancing vaginal pH by eliminating bad bacteria, viruses, and protozoa, thereby allowing good bacteria to re-colonize.

The basic components of the present invention are as follows:

1. ½ tsp chicken foot powder made from finely ground chicken feet cooked in a small pit covered with soaked leaves of collard greens for 7 hours.

2. 1 cup cranberries—prepared by boiling 1 cup of berries in conch pea and watermelon (all parts) juice, distilled water and amaranth seed, and snake root.

3. Lemon leaves and 1 whole lemon. Lemon has been boiled for 3 hours in a protein solution made from beef oxtails juice and oxtail bone ground. 1 tsp of ground bone.

To make 1 gallon of the present invention:
Ingredients
   a. ¼ tsp ground chicken foot powder.
   b. ⅛ tsp ground oxtail bone powder.
   c. ⅓ cup conch peas.
   d. ¼ tsp. amaranth seed oil.
   e. 1/10 tsp. snake root leaf powder.
   f. 5 cups cranberry (all species Vaccinum).
   g. 10 oranges (all species).
   h. 2 lemons (all species).
   i. 1 cup pomegranate juice.
   j. ½ tsp brassica powder.
   k. ½ carrot powder.
   l. 2 Bunches collard greens.
   m. Hibiscus leaf oil ⅛ tsp.
   n. Watermelon 2 cups.
   o. Malic acid, potassium sorbate, citric acid, glycerin, sorbitol.
Prep. A
   1. Stuff cranberries, blueberries inside of oranges and lemons after cleaning out the middle portion of the citrus fruit, after removing the top of the citrus fruit.
   2. Prepare a water bath and submerge citrus fruit leaving the opened end outside of the bath.
   3. Cook at medium heat 324 F.° for 7 hours.
   4. Remove from water.
   5. Place the bottom portion the sieve into the bottom end of the citrus. Then, cut off the bottom portion of the citrus.
   6. Insert the top portion of the sieve unexpanded.
   7. Then expand the sieve and twist 3 times.
   8. Remove the top portion of the sieve and squeeze the juice through the bottom portion of the sieve.
   9. Set juice aside.
Prep. B

1. Take 1 medium watermelon. Cut off top and remove cups of the meat of the watermelon.

2. Wrap watermelon in aluminum foil, leaving the top exposed.

3. Add into the watermelon ⅓ cup conch peas and liquid A. juice.

4. Submerge watermelon into a water bath 250 F* for 3 hours.

5. Take hand mixer and mix the watermelon mixture until thoroughly mix.

6. Pour mixture through a large sieve and collect juice. set aside.
Prep. C
   1. Prepare a large pot of water.
   2. Add collard greens,½ tsp brassica powder, 1/10 tsp. snake root leaf powder, 2 cups of watermelon meat,½ carrot powder, hibiscus leaf oil ⅛ tsp., ¼ tsp of chicken foot powder, ⅛ tsp oxtail bone ground up.
   3. Cook for 3 hours.
Last
   1. Mix 30 mL of Prep. C, 1,180 mL of Prep. B, 1,200 mL of pomegranate juice, and
   1,375 mL of Prep. A thoroughly.

The complete list of components in the present invention are as follows:
Species Citrus×aurantiifolia (Christm.) Swingle (pro sp.)—key lime P.
Species Citrus×aurantium L. (pro sp.)—sour orange P
   Subspecies Citrus×aurantium L. ssp. aurantium—sour orange P.
   Subspecies Citrus×aurantium L. ssp. bergamia (Risso & Poit.) Wight & Am. ex Engl.—bergamot orange P.
Species Citrus hystrix DC. P.
Species Citrus jambhiri Lush.—rough lemon P.
Species Citrus limetta Risso—bitter orange P.
Species Citrus×limon (L.) Burm. f. (pro sp.)—lemon P.
Species Citrus×limonia Osbeck (pro sp.)—Mandarin lime P.
Species Citrus macroptera Lour. P. 5
Species Citrus maxima (Burm. f.) Merr.—shaddock P.
Species Citrus medica L.—citron P.
Species Citrus nobilis Lour.—tangor P.
Species Citrus×paradisi Macfad. (pro sp.)—grapefruit P.
Species Citrus reticulata blanco—tangerine P
   Subspecies Citrus reticulata blanco ssp. unshiu (Marcow.) D. Rivera Núñez et al.—Unshu orange P.
Species Citrus×sinensis (L.) Osbeck (pro sp.)—sweet orange P.
Species Citrus tachibana (Makino) T. Tanaka—tachibana orange P.
Species Citrus×tangelo J. W. Ingram & H. E. Moore—tangelo P.
Vaccinium arboreum—Sparkleberry.
Vaccinium crassifolium—Creeping Blueberry.
Sect. Brachyceratium
   Vaccinium dependens.
Sect. Bracteata
   Vaccinium acrobracteatum.
   Vaccinium barandanum.
   Vaccinium bracteatum.
   Vaccinium coriaceum.
   Vaccinium cornigerum.
   Vaccinium cruentum.
   Vaccinium hooglandii.
   Vaccinium horizontale.
   Vaccinium laurifolium.
   Vaccinium lucidum.
   Vaccinium myrtoides.
   Vaccinium phillyreoides.
   Vaccinium reticulatovenosum.
   Vaccinium sparsum.
   Vaccinium varingifolium.
Sect. Ciliata
   Vaccinium ciliatum.
   Vaccinium oldhamii.
Sect. Cinctosandra
   Vaccinium exul.
Sect. Conchophyllum
   Vaccinium corymbodendron.
   Vaccinium delavayi.
   Vaccinium emarginatum.
   Vaccinium griffithianum.
   Vaccinium meridionale.
   Vaccinium moupinense—Himalayan Blueberry.
   Vaccinium neilgherrense.
   Vaccinium nummularia.
   Vaccinium retusum.
Sect. Cyanococcus—typical blueberries
   Vaccinium angustifolium—Lowbush Blueberry.
   Vaccinium boreale—Northern Blueberry.
   Vaccinium caesariense—New Jersey Blueberry.
   Vaccinium caespitosum—Dwarf Blueberry (Dwarf Bilberry).
   Vaccinium corymbosum—Highbush Blueberry.
   Vaccinium darrowii—Evergreen Blueberry.
   Vaccinium elliottii—Elliott's Blueberry.
   Vaccinium formosum.
   Vaccinium fuscatum—Black Highbush Blueberry; syn. V. atrococcum.
   Vaccinium hirsutum.
   Vaccinium koreanum—Korean Blueberry.
   Vaccinium myrsinites—Evergreen Blueberry.
   Vaccinium myrtilloides—Canadian Blueberry.
   Vaccinium pallidum Ait.—Dryland Blueberry (images); syn. V. vacillans Torr.
   Vaccinium simulatum.
   Vaccinium tenellum.
   Vaccinium virgatum—Rabbiteye Blueberry; syn. V. ashei.
Sect. Eococcus
   Vaccinium fragile.
Sect. Epigynium
   Vaccinium vacciniaceum.
Sect. Galeopetalum
   Vaccinium chunii.
   Vaccinium dunalianum.
   Vaccinium glaucoalbum.
   Vaccinium urceolatum.
Sect. Hemimyrtillus
   Vaccinium arctostaphylos.
   Vaccinium cylindraceum.
   Vaccinium hirtum.
   Vaccinium padifolium.
   Vaccinium smallii.
Sect. Myrtillus (including sect. Macropelma)—bilberries and relatives
   Vaccinium calycinum Sm.—Ōhelo kau la'au (Hawai'i).
   Vaccinium cereum (L.f.) Forst.f.—East Polynesian Blueberry, Pacific Blueberry.
   Vaccinium cespitosum—Dwarf Bilberry.
   Vaccinium deliciosum—Cascade Bilberry, Cascade Blueberry, Blueleaf Huckleberry.
   Vaccinium dentatum Sm.—Ōhelo (Hawai'i).
   Vaccinium membranaceum—Square-twig Blueberry, Thinleaf Huckleberry, Tall Huckleberry, Big Huckleberry, Mountain Huckleberry, “black huckleberry”.
   Vaccinium myrtillus—Common Bilberry, Blue Whortleberry, Blaeberry, Fraughan, Hurtleberry.
   Vaccinium ovalifolium—Alaska Blueberry, Early Blueberry, Oval-leaf Blueberry.
   Vaccinium parvifolium—Red Huckleberry.
   Vaccinium praestans —Krasnika (Russian: κpacH [symbol] κa).
   Vaccinium reticulatum—Ōhelo 'ai (Hawai'i).
   Vaccinium scoparium—Grouse Whortleberry, Grouseberry, Littleleaf Huckleberry.
Sect. Neurodesia
   Vaccinium crenatum.
Sect. Oarianthe
   Vaccinium ambyandrum.
   Vaccinium cyclopense.
Sect. Oreades
   Vaccinium poasanum.
Sect. Pachyanthum
   Vaccinium fissiflorum.
Sect. Polycodium
   Vaccinium stamineum L.—Deerberry; syn. V. caesium (Eastern North America).
Sect. Pyxothamnus
   Vaccinium consanguineum.
   Vaccinium floribundum.
   Vaccinium ovatum Pursh—California Huckleberry (or Evergreen Huckleberry) (Coastal Western North America).
   Sect. Vaccinium
   Vaccinium uliginosum L.—Northern (or Bog) Bilberry (or Blueberry); syn. V. occidentale (Northern North America and Eurasia).
Sect. Vitis-idaea
   Vaccinium vitis-idaea L.—Partridgeberry, Cowberry, Redberry, Red 5 Whortleberry, or Lingonberry (northern North America and Eurasia).
Citrus×aurantiifolia (Christm.) Swingle (pro sp.) [medica×sp.] key lime .
LIAU7 Limonia aurantifolia Christm., orth. var.
LIAU9 Limonia aurantiifolia Christm.
CILI5 Citrus×limon (L.) Burm. f (pro sp.) [medica×aurantifolia] lemon.
CILI Citrus limon (L.) Burm. f., database artifact.
CILI3 Citrus×limonia Osbeck (pro sp.) [limon×reticulata] Mandarin lime.
CILI4 Citrus limonum Risso.
LIMON2 Limonia L. limonia.
LIACS Limonia acidissima L. Indian woodapple.
FELI3 Feronia limonia (L.) Swingle.
LIMON Limonium Mill. sea lavender.
LIAR10 Limonium arborescens (Brouss.) Kuntze tree limonium.
LIBR3 Limonium brassicifolium (Webb & Berthel.) Kuntze.
LIBI4 Limonium binervosum (G. E. Sm.) C. E. Salmon rock sea-lavender.
LICAS Limonium californicum (Boiss.) A. Heller marsh rosemary.
LICAM2 Limonium californicum (Boiss.) A. Heller var. mexicanum (S. F. Blake) Munz.
LIME6 Limonium mexicanum S. F. Blake.
LICA17 Limonium carolinianum (Walter) Britton lavender thrift.
LIAN9 Limonium angustatum (A. Gray) Small.
LICAA2 Limonium carolinianum (Walter) Britton var. angustatum (A. Gray) S. F. Blake.
LICAA3 Limonium carolinianum (Walter) Britton var. angustifolium S. F. Blake.
LICAC4 Limonium carolinianum (Walter) Britton var. compactum Shinners.
LICAN2 Limonium carolinianum (Walter) Britton var. nashii (Small) B. Boivin.
LICAO Limonium carolinianum (Walter) Britton var. obtusilobum (S. F. Blake) H. E. Ahles.
LICAT3 Limonium carolinianum (Walter) Britton var. trichogonum (S. F. Blake) B. Boivin.
LINA Limonium nashii Small.
LINAA Limonium nashii Small var. angustatum (A. Gray) H. E. Ahles.
LINAA2 Limonium nashii Small var. albiflorum (Raf.) House.
LINAT Limonium nashii Small var. trichogonum (S. F. Blake) S. F. Blake.
LIOB3 Limonium obtusilobum S. F. Blake.
LITR8 Limonium trichogonum S. F. Blake.
LIGE3 Limonium gerberi Soldano.
LILA12 Limonium latifolium (Sm.) Kuntze, nom. illeg.
LILE8 Limonium leptostachyum (Boiss.) Kuntz statice.
LILI4 Limonium limbatum Small Trans-Pecos sea lavender.
LILIG Limonium limbatum Small var. glabrescens Correll.
LIOT Limonium otolepis (Schrenk) Kuntze saltmarsh sea lavender.
LIPE Limonium perezii (Stapf) F. T. Hubbard Perez's sea lavender.
LIRA2 Limonium ramosissimum (Poir.) Maire Algerian sea lavender.
LIRAP Limonium ramosissimum (Poir.) Maire ssp. provinciale (Pign.) Pign. 20 Algerian sea lavender.
LIRE14 Limonium reniforme (Girard) Linchevskii.
LIPE3 Limonium perfoliatum (Karelin ex Boiss.) Kuntze.
LISI2 Limonium sinuatum (L.) Mill. wavyleaf sea lavender.
LIVU3 Limonium vulgare Mill. Mediterranean Sea lavender.
STLI8 Statice limonium L.
LLIMO Llimoniella Hafeller & Nav.-Ros.
LLNE Llimoniella neglecta (Vain.) Triebel & Rambold.
SEPU Sedum pulchellum Michx. Widowscross.
CHVI16 Chetyson vigilimontis (Small) Á. Löve & D. Löve.
SEVI8 Sedum vigilimontis Small.
Brassica oleracea (collard greens).
Citrullus Schrad. Watermelon.
Citrullus lanatus (Thunb.) Matsum. & Nakai watermelon.
Citrullus lanatus (Thunb.) Matsum. & Nakai var. citroides (L. H. Bailey) Mansf. Watermelon.
Citrullus lanatus (Thunb.) Matsum. & Nakai var. lanatus watermelon.
Solanum citrullifolium A. Braun watermelon nightshade.
Solanum citrullifolium A. Braun var. citrullifolium watermelon nightshade.
Solanum citrullifolium A. Braun var. setigerum Bartlett watermelon nightshade.
Watermelon juice.
Ground chicken feet.
Lemon leaves.
Snakeroot.
Amaranth seed.
Ground oxtail bone.
Lemon leaf.
Cranberry vine leaf.
Distilled water.

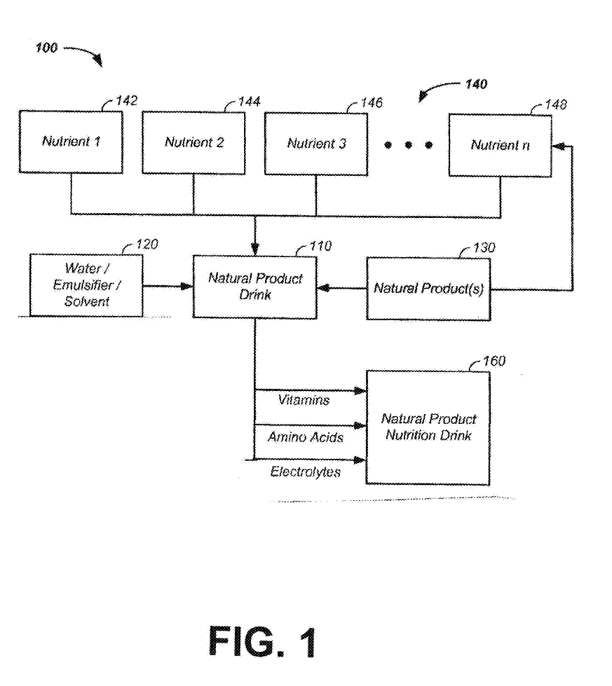

Referring now to FIG. 1, a flow chart illustrating formation of a natural product drink 100 for balancing vaginal pH, reducing vaginal odor and/or reducing body odor is provided.

Still referring to FIG. 1, herein a natural product drink 110 is a combination of a solvent 120, a natural product 130, and/or a nutrient 140. The solvent 120 is any liquid, aqueous mix, non-aqueous based solvent, water, an emulsifier, and/or a suspension agent. Generally, a natural product is chemical compound or substance produced by a living organism. A natural product can be considered as such even if it can be prepared by total synthesis.

Herein, a natural product 130 is a chemical, chemical compound, or substance produced by a living organism found in nature. The natural product 130 is produced by a plant or animal. Herein, a nutrient 140 is a product derived from a natural product, is a constituent of a natural product, or is a simulant of a constituent of a natural product. An artificially produced chemical simulant of a natural product nutrient is a chemical, substance, and/or mixture that retains one or more properties of the nutrient 140, but is man-made. The natural product drink 110 optionally contain 1, 2, 3, . . . n nutrients 142, 144, 146, 148, where n is a positive integer.

Still referring to FIG. 1, a natural product drink 110 is optionally combined with: (1) vitamins to form a vitamin enhanced natural product drink, (2) amino acids to form an amino acid enhanced natural product drink or protein enhanced natural product drink, or (3) electrolytes to form an electrolyte enhanced natural product drink. Generally, the natural product drink 110 is optionally supplemented with vitamins, amino acids (such as L-leucine), electrolytes, and/or an additive to form a natural product nutrition drink 160.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

Although the invention has been explained in relation to its preferred embodiment, it is to be understood that many other possible modifications and variations can be made without departing from the spirit and scope of the invention.

1. A composition for balancing vaginal pH consisting essentially of therapeutic amounts of watermelon juice, ground chicken feet, lemon leaves, snakeroot, Amaranth seed, ground oxtail bone, cranberry vine leaf, and distilled water.

2. The composition of claim 1, wherein the composition corresponds to a pill.

3. The composition of claim 1, wherein the composition corresponds to a gel.

4. The composition of claim 1, wherein the composition corresponds to a powder.

5. The composition of claim 1, wherein the composition corresponds to a liquid.

6. An aqueous beverage for balancing vaginal pH wherein a gallon serving comprises:
¼ tsp ground chicken foot powder;
⅛ tsp ground oxtail bone powder;
⅓ cup conch peas;
¼ tsp. amaranth seed oil;
1/10 tsp. snake root leaf powder;
5 cups cranberry;
10 oranges;
2 lemons;
1 cup pomegranate juice;
½ tsp brassica powder;
½ carrot powder;
2 Bunches collard greens;
⅛ Hibiscus leaf oil;
2 cups of Watermelon;
Malic acid;
potassium sorbate;
citric acid; glycerin;
and sorbitol.
